(19) United States
(12) Patent Application Publication
(10) Pub. No.: US 2010/0191270 A1
(43) Pub. Date: Jul. 29, 2010

(54) INTRAGASTRIC BALLOON ASSEMBLY

(76) Inventor: Jose Rafael Garza Alvarez, Mexico City (MX)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 12/303,701

(22) PCT Filed: Jun. 5, 2006
(86) PCT No.: PCT/MX06/00044

§ 371 Date: Jan. 12, 2009

(51) Int. Cl. A61M 29/02; A61B 6/12

(52) U.S. Cl. 606/192; 600/424

(57) ABSTRACT

The invention relates to an intragastric balloon for the treatment of persons having severe or morbid obesity problems, wherein the number of elements in the already known systems is reduced, and provides a material savings in the manufacture thereof. The intragastric balloon assembly comprises a balloon connected to a catheter to inflate the same, and includes a means for fixing the same to a patient body, consisting in a plain fixing plate with a sole orifice through which said inflation catheter is passed, to be fixed subcutaneously in the abdominal region of said patient; and in a preferred embodiment, an additional fastening element is provided allowing firmly subject said inflation catheter to said fixing plate, and consisting in a semi-rigid washer or ring made of silicon, which is fastened to the catheter and fixing plate by means of a suitable adhesive; thus maintaining said catheter with a pulling suitable for the balloon to be pasted perfectly but without pressure at all, on the stomach wall. Said assembly also comprises a multi-puncture valve to which said inflation catheter is connected to inflate said balloon, which in turn can be arranged on the fixing plate, whereby an alternative element to fasten the catheter and, therefore, the balloon assembly is obtained.

### FIELD OF THE INVENTION

This invention has application to the gastroenterology and general surgery fields, specially for the treatment of persons suffering of severe or morbid obesity.

### BACKGROUND OF THE INVENTION

During the last two decades several intra and extra gastric devices have been developed as a help in reducing the weight of persons suffering of severe or morbid obesity. The presently more developed devices include bands and balloons, which have been subjected to various modifications along that time in order to improve the efficiency thereof and avoid complications; however, such problems as gastric juices leaks and infection, necrosis of the gastric wall due to the contact with said devices, etc., have not been solved, as well as those general situations representing problems for patients and frequently failures or non-efficient results regarding the weight loss. Likewise, the surgical techniques used for treating severe obesity are complicated and mean an morbidity and mortality increase aside of the high cost for these treatments.

An example of the devices of said type can be found in the U.S. patent application No. US 2003/0158569 A1, with a publication date of Aug. 21, 2003, of Hussein Wazne, wherein an intragastric device is disclosed for treating morbid obesity, to be inserted through endoscopic technique through the mouth of the patient, and comprising a balloon or envelopment having a specific nominal volume connected in a sealed way to connecting means comprising a disc capable of forming a support base for the balloon against the wall of the stomach; a flexible tube or catheter to connect said balloon to a filling device, and fastening means integral with the tube or catheter, providing said connecting means the position or removing of said balloon and the situation thereof either inside the stomach of the patient or subcutaneously, by filling said balloon up to a predetermined nominal volume. Said document does not specify the material from which said device is formed; it is only described that it is of plastics, such as polypropylene or of silicon, without further details. On the other side, it is described that the balloon can be inflated within a range of from 100 cc up to 1000 cc, a too wide range for the balloon elasticity, which points to the inflation with air or a liquid, indistinctly.

Likewise, it is mentioned that said device has two fixing plates, one inside the stomach, in order to prevent possible leaks by pressing said plate into the internal wall of the stomach; and another outer fixing plate, towards the aponeurosis or either towards the skin, but there is no indication on how to locate or fixe the same.

One of the disadvantages of said document is that an incision must be carried out on the patient during the fixing of the balloon in order to extract the catheter; and a great care is to be taken in said zone to perfectly seal said aperture, aside of the use of solutions or drugs to avoid an infection or necrosis of tissues due to the contact thereof with the fixing means. It is not explained how this device is to be situated; although endoscopy is mentioned to introduce said balloon, no description of the manner to carry this out is given. Also no mention is made of how to introduce said catheter through the abdominal wall down to the stomach. Other important aspect to be mentioned on the above identified US patent application is that elements and characteristics very similar to a device covered by Mexican patent No. 234,516 jointly owned by the inventor of this application, are disclosed; wherein only the fixing axis is retired leaving said catheter as such with two plates, one of which is internal. This, instead of solving the problem of intragastric liquids leaks towards the abdominal wall, can increase them or be the origin of others, since the pressure of said plate onto the mucosa and the gastric wall can five place to injuries that can be from an irritation and ulceration, up to necrosis of the tissues.

On the other side, a patent document WO 02/40081 A2, Thomas Bales et al, describes an intragastric balloon device suitable to vary its volume repeatedly without external help, consisting in a inflation/deflation system, externally programmable, by means of which said balloon can take a desired size and can be situated within the stomach cavity. Said document does not describe fixing elements to prevent the migration of the balloon within the digestive tract, which can originate greater complications, and also no elements to avoid inconveniences after the situation of the balloon in place.

U.S. Pat. No. 6,755,869, Kart Geitz describes an intragastric prosthesis for treating morbid obesity, consisting of a porous fabric made of bioabsorbing filaments having an open mesh configuration, formed in a flattened shape having dimensions greater than the esophagi aperture and the gastric outlet in the stomach; with said prosthesis being unfolded within the stomach and being of a size capable of being retained at the immediate portion thereof so as to exert a pressure on the upper fundus. No exact mention can be found in said document on whether there are fixing elements for the prosthesis, and according to the disclosure, said prosthesis can move from side to side within the stomach, thus provoking a major problem, such as the obstruction of any of the food inlet or outlet zones.

Regarding Mexican patent No. 234,516, owned by the same inventor of the present, an intragastric balloon is described, combining three elements: a valve to control the inflation and fixing means for the assembly, thus preventing the migration of the balloon inside the stomach. Although the assembly proposed therein provides an arrangement efficient for fixing said balloon in a secure manner, the number of elements required makes that the assembly is of relatively complicated manufacture, position and manipulation and of high cost.

### SUMMARY OF THE INVENTION

This invention is aimed to provide an intragastric balloon assembly with simple design and construction, to simplify the manufacture thereof and reduce the cost in connection therewith.

Thus, it is an object of this invention to provide an intragastric balloon assembly capable of remain fixed within a patient stomach, on a pre-established site, during the period of treatment thereof, without major complications, by fixing elements, wherein said balloon can be inflated with a fluid, such as a gas or environment air.

It is also an object of this invention to provide a means to fasten firmly the catheter to a fixing plate and thus maintain fixed the intragastric balloon assembly.

It is another object of this invention to provide the balloon assembly with elements capable of preventing possible leakages of intragastric liquid towards soft tissues at the abdominal wall.

Another further object of this invention is to provide a means allowing the control of the balloon volume in a simple manner and without major discomfort for the patient.

These and other objects are attained by the intragastric balloon assembly of this invention, wherein the need of a rigid support, as disclosed in Mexican patent No. 234,516, of the same inventor as the present, is avoided, and the function of which is carried out by the inflation catheter itself, arranged in one of the poles of the balloon, formed unitary thereto.

The fixing system is comprised of a plain fixing plate with a sole orifice through which an inflation catheter is passed; said plate is subcutaneously fixed at the patient abdominal region and, in a preferred embodiment, it is provided also an element to fasten the inflation catheter firmly to said fixing plate, and consisting of a semi-rigid silicon sheave or ring, including a small rim; said sheave or ring is fixed with an appropriated adhesive thus provoking that a suitable stress of the catheter is obtained so as said balloon remains perfectly joined, but without pressure at all, on the stomach wall; whereby the tissue becoming in contact with the material of the balloon assembly is prevented from necrosis in said zone and, at the same time, a firm fastening of the catheter and, therefore, of the assembly is obtained, thus allowing that the same remains fixed in the desired position.

In order to prevent leakages of intragastric liquid towards the soft tissues of the abdominal wall, the invention provides on that portion of the catheter located at the exit of the balloon, and for about 3 cm of the length thereof, a film of a material capable of promoting the fibrosis and the healing of the tissue, such as goretex or other similar substances.

On the other side, in order to inflate the intragastric balloon and control the volume thereof, following the positioning thereof within the patient, a multi-puncture valve is provided, connected to the balloon inflation catheter, which, in turn, can be arranged on the fixing plate, thus having an alternative element to fasten the catheter and maintain the same under stress and, thus, maintain the balloon assembly fixed therein. All of these elements become arranged under the skin and the subcutaneous cellular tissue.

In an additional embodiment said multi-puncture valve can be arranged farther from the plate, by making another small incision in the skin and forming a tunnel at the cellular tissue, in order to pass said catheter therethrough and connect it to said valve.

The present invention seeks to lower the number of elements in those already known balloon systems, and obtain material savings during the manufacture thereof; whereby lower manufacture costs, and easier and faster positioning can be obtained; and also the costs of possible inflation modification will be undoubtedly reduced, since the endoscopic procedure and the anesthetic sedation are suppressed.

### BRIEF DESCRIPTION OF FIGURES

The invention is illustrated by means of the enclosed exemplary drawings.

FIG. 1 shows the intragastric balloon assembly of this invention, in an exploded manner or as separated apart pieces.

FIG. 2 shows a preferred embodiment of the intragastric balloon assembly, without the fixing elements.

FIG. 3 shows the assembling manner of the intragastric balloon assembly with the fixing elements; and

FIG. 4 is an schematic view showing how the intragastric balloon remains arranged within the body of a patient.

### DETAILED DESCRIPTION OF THE INVENTION

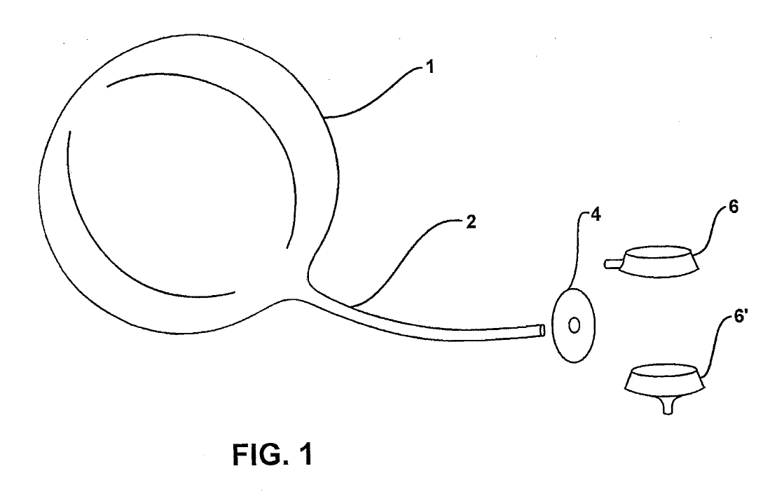

The present invention is comprised of an intragastric balloon assembly, which, as shown in FIG. 1, includes a balloon 1 of a material such a biomedical grade silicon with a volume capacity of 250 700 cm³; an inflation catheter 2, formed unitarily with the balloon, having a length of about 100 cm and a diameter between 1.5 and 2.0 mm, arranged in one of the poles of said balloon 1, through which a fluid or air can be introduced so as to inflate said balloon 1.

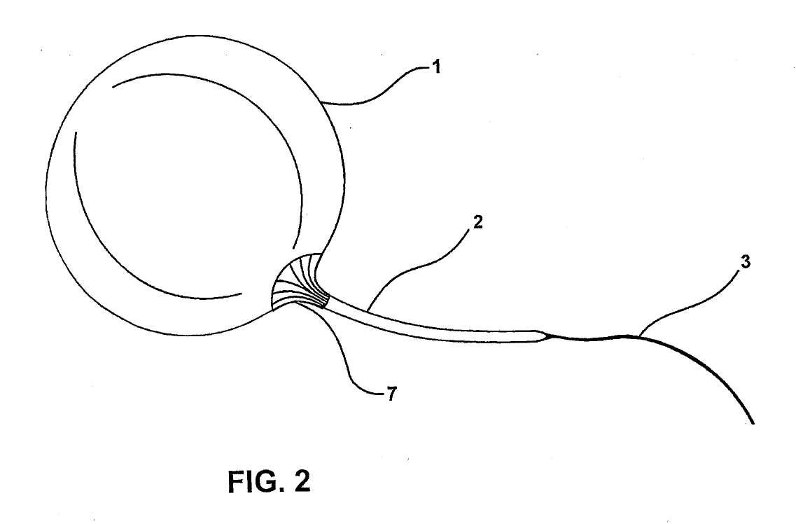

In a preferred embodiment, illustrated in FIG. 2, said inflation catheter 2 is provided with a pulling guide 3 as an extension at the distal end thereof to said balloon 1; said guide will be used to pull the balloon into the gastric cavity, and to pass said catheter 2 through the stomach wall and other abdominal tissues. Said pulling guide 3 can be made of a material such a silicon more resistant or polypropylene.

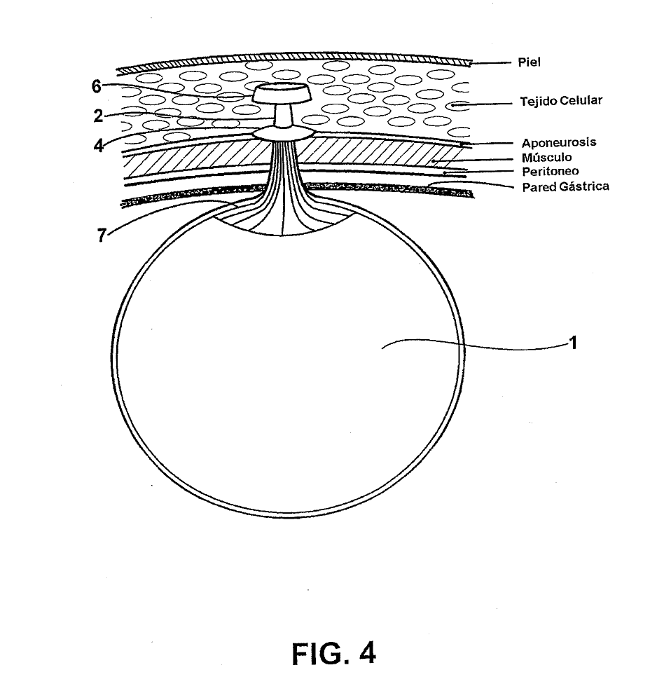

The assembly of the invention includes also a fixing plate 4, that is a generally plain plate preferably made of a medical grade material, such as silicon, and preferably having a thickness of from 1.5 to 2.0 mm, with a preferred diameter of from 4 and 4 cm, and having an orifice through which said inflation catheter 2 is passed. Said fixing plate 4 is positioned and secured on the anterior aponeurosis of the straight muscles of the abdomen, as shown in FIG. 4, at the site previously determined by endoscopy, and once the inflation catheter 2 taut, it is secured into the fixing plate 4, by means of a suitable adhesive, thus providing a catheter suitable stress, so that the balloon becomes perfectly connected, but without pressure at all, on the wall of the stomach; thus avoiding that the tissue becoming in contact with the material of the balloon assembly be affected by necrosis on said zone and, at the same time, that a firm fastening of the catheter is provided and therefore, of the assembly in general, whereby the latter can be maintained firm in the desired position. In this form, said inflation catheter 2, together with the fixing plate 4 serves as the fixing means for said balloon 1 into the abdominal wall.

According to a preferred embodiment of the invention, a film 7 is provided of a material promoting the increase of fibrosis and healing, such as goretex or other similar substances, at the balloon 1 zone in contact with the abdominal wall, as well as on a portion of said catheter 2, preferably for a length of about 3 cm at the end connected to said balloon 1, as shown in FIGS. 2 thru 4, thus securing the adhesion of the edges of the perforation made at the gastric wall, to the balloon assembly, and thus preventing leakages of intragastric liquids towards the soft tissues, through the abdominal wall perforation.

Furthermore, the balloon assembly of this invention comprises a multi-puncture valve 6 for inflation and control of volume of said balloon 1, to be connected to the distal end of said inflation catheter 2; whereby said balloon 1 can be initially inflated by means of the valve once said balloon is arranged inside the stomach of the patient, and the volume of inflation of said balloon 1 can be modified during the treatment of the patient, according to the results obtained during the treatment; i.e., as per the weight reduction of the patient.

Said multi-puncture valve 6, according to an embodiment of the invention, is connected to the inflation catheter 2 in a place immediate to the site wherein said fixing plate is arranged; in such a form that said inflation catheter 2 can be maintained firm, helping to the fixing and maintaining of said balloon 1 within the stomach of the patient, in a way such as illustrated in FIG. 4.

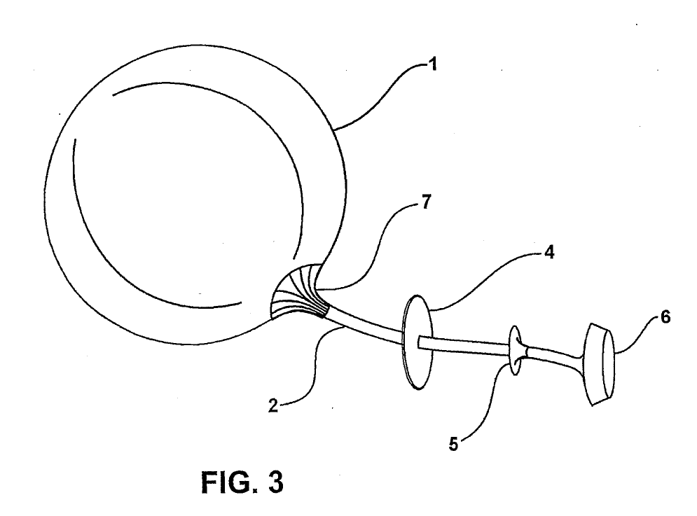

In an alternative embodiment, a washer or fixing ring 5 is provided, shown in FIG. 3, provided with an orifice with a rim through which said inflation catheter is introduced and is adhered thereto by means of a suitable adhesive, thus providing for the desired fixing of said elements, in order to maintain said intragastric balloon 1 in a fixed position inside a patient stomach.

Once the inflation catheter 2 fixed with assistance of the fixing plate 4 and said fixing ring 5, the inflation valve 6 can be arranged in a remote site relating said fixing plate 4, carrying the inflation catheter subcutaneously up to the connection thereof to said valve 6.

For evaluation and volume control of the inflation of said balloon 1 of the present invention, in another embodiment, there is provided additionally a coating on said balloon 1 or radio-opaque markers that can be metal filaments included within the silicon (not shown) or other similar systems allowing the identification thereof by means of radiological techniques. In this manner, with a simple X-ray technique and local anesthetics, the volume of the balloon inside the patient can be appreciated and a modification thereof can be carried out by effecting a puncture with a needle on said valve 6 either for deflating or inflating said balloon 1, thus avoiding the need of an endoscopy technique for said evaluation and control.

The surgical technique consists in a combination of the endoscopy procedure of gastrostomy, and the use of the intragastric balloon of the present invention, and comprises the steps of:

A) puncturing the abdominal wall in a predetermined site with the endoscope, with a 2 mm trocar, thus reaching the interior of the stomach, which has been inflated previously by means of a puncture on the gastric wall, down to the abdominal wall;

B) carrying out previously on the site an incision of skin and cellular tissue down to the aponeurosis;

C) introducing the inflation catheter 2 of the intragastric balloon assembly by its distal end, into the inner cavity of the stomach, by means of an endoscope, by holding said end of the catheter 2 with a clamp passed through said 2 mm trocar; once said catheter is passed, the same is extracted upon retiring the trocar up to the abdominal wall, wherein the pulling thereof can be effected, and at the same time, the pulse of the balloon that is situated at the other end of the catheter, by means of the same endoscope, so as to be able to push and pull it until the final position thereof within the stomach;

C) retract said catheter into the abdominal wall;

D) urging said catheter towards the incision zone, until said balloon (continuously monitored by the endoscope) becomes contiguous to the gastric wall, but without the use of a great stress, so as to avoid the possibility of a necrosis at said wall;

E) placing the fixing plate 4, by passing said inflation catheter 2 through the orifice of the plate and supporting the entire intragastric balloon system;

F) connecting the distal end of said catheter 2 to the multi-puncture valve 6, in a zone immediate to the site wherein said fixing plate 4 is arranged, in such a manner that the inflation catheter 2 is maintained taut and fastened and, therefore, the fixing and support of said balloon 1 is obtained within a patient stomach.

In an alternative embodiment, the following steps are included:

F′) placing a fixing washer 5, by passing the inflation catheter 2 through the orifice thereof, with said fixing washer 5 arranged on said fixing plate 4;

G) adhering the fixing elements (4 and 5) with a surgical adhesive, thus avoiding the use of suture stitches or metal staples;

H) connecting the distal end of said catheter to the multi-puncture valve 6, and fixing said valve 6 subcutaneously.

In another further embodiment, when the surgeon judges appropriated, the following can be carried out:

HI′) connecting the inflation catheter 2 to the multi-puncture valve 6 by moving it to a side, thus positioning said valve 6 in a site farther from said fixing elements 4 and 5, making another small incision in the skin and tunneling the cellular tissue in order to pass said catheter 2 and connect the same to said valve 6.

By means of this arrangement, the intragastric balloon assembly seeks to reduce the number of elements employed in the balloons actually in the market and those described in the prior art. Moreover, this arrangement reduces the number of pieces to be manufactured for this purpose, by employing multi-puncture valves, that are elements available in the market and are easy to obtain, whereby the production costs will be lower. Likewise, the placing will be more rapid and simple, and also the cost of any possible modification of inflation undoubtedly will be lower, as this is carried out by radiological control only, without hospitalization, suppressing the endoscopic procedure otherwise indispensable with the balloon assembly of the prior patent of the same inventor.

It is important to note that the efficiency in the use of the present invention, regarding the results as a reduction in the weight of the patients is equivalent to that of the surgical procedures or that of the gastric band, but without the trauma of any of said procedures; and said efficiency is enormously greater than that of the present balloons, only recommended for temporal treatments and with low effectiveness regarding the tolerance and the loss of weight.

It will be apparent for a person with mean knowledge in the art that several modifications and adaptations can be effected in the invention as described and illustrated herein, without coming out of the spirit and the reach thereof.

1. An intragastric balloon assembly of the type comprising an intragastric balloon, a pulling element to carry the balloon into the gastric cavity of a patient through the mouth and gastric tube thereof; an inflation catheter connected to the balloon, to be passed through the stomach wall and abdominal tissues, to inflate therethrough the intragastric balloon with a fluid; fixing elements to fixe the balloon in a predetermined position within the gastric cavity of the patient, thus avoiding the migration of the balloon; and an element to control the inflation volume of the intragastric balloon; wherein the inflation catheter is formed unitarily with the balloon and has a distal end free that serves as a pulling element; the fixing elements consist in a fixing plate with a sole orifice through which the distal end of the inflation catheter is passed, with the plate being fixed on the anterior aponeurosis of the straight muscles of the abdomen; and an element for the control of the inflation volume, consisting in a multi-puncture valve of inflation, to be connected to the distal end of the inflation catheter, in a place posterior to the fixing plate, and to be fixed under the skin of the patient at the abdominal region.

2. Intragastric balloon assembly according to claim 1, additionally comprising a pulling guide, formed as a projection of the distal end of the inflation catheter, in order to effect the pulling of the balloon towards the gastric cavity and carry the inflation catheter through the stomach wall and other abdominal tissues.

3. Intragastric balloon assembly according to claim 1, wherein the inflation multi-puncture valve is arranged connected to the inflation catheter at a place immediate to the site wherein the fixing plate is disposed, thus serving as a fastening element to fix the inflation catheter to the fixing plate.

4. Intragastric balloon assembly according to claim 1, further comprising a fixing washer having an orifice with a rim, through which the inflation catheter is passed; with the fixing washer arranged on the fixing plate, thus allowing to arrange the multi-puncture valve at a place far from the fixing plate.

5. Intragastric balloon assembly according to claim 1, further comprising a film of a material capable of increasing the fibrosis and healing in the zone of the balloon and catheter becoming in contact with the abdominal wall.

6. Intragastric balloon assembly according to claim 1, wherein the intragastric balloon comprises additionally a radio-opaque coating for radiologic evaluation of the balloon disposed within a patient.

7. Intragastric balloon assembly according to claim 1, wherein the intragastric balloon comprises additionally radio-opaque markers for radiologic evaluation of the balloon disposed within a patient.

8. Intragastric balloon assembly according to claim 7, wherein the radio-opaque markers are metal filaments included in the silicon.

9. Method to implant an intragastric balloon assembly in a patient suffering of obesity, of the type employing endoscopic techniques, comprising the steps of:
A) puncturing the abdominal wall in a site predetermined by the endoscope, with a 2 mm trocar reaching the interior of the stomach that has been previously inflated, by a puncture of the gastric wall, down to the abdominal wall;
B) making previously an incision at the skin and cellular tissue site up to the aponeurosis;
C) introducing the inflation catheter of the intragastric balloon assembly by its distal end, into the inner cavity of the stomach, by means of an endoscope, by holding the end of the catheter with a clamp passed through the 2 mm trocar; once the catheter is passed, the same is extracted upon retiring the trocar up to the abdominal wall, wherein the pulling thereof can be effected, and at the same time, the pulse of the balloon that is situated at the other end of the catheter, by means of the same endoscope, so as to be able to push and pull it until the final position thereof within the stomach;
C) withdrawing the catheter into the abdominal wall;
D) urging the catheter towards the incision zone, until the balloon continuously monitored through the endoscope becomes contiguous to the gastric wall, but without the use of a great stress, so as to avoid the possibility of a necrosis at the wall;
E) placing the fixing plate, by passing the inflation catheter through the orifice of the plate and supporting the entire intragastric balloon system; and
F) connecting the distal end of the catheter to the multi-puncture valve, in a zone immediate to the site wherein the fixing plate is arranged, in such a manner that the inflation catheter is maintained taut and fastened and, therefore, the fixing and support of the balloon 1 is obtained within a patient stomach.

10. Method according to claim 8, comprising the alternative steps of:
F′) placing a fixing washer, by passing the inflation catheter 2 through the orifice thereof, with the fixing washer arranged on the fixing plate;
G) adhering the fixing elements with a surgical adhesive, and
H) connecting the distal end of the catheter to the multi-puncture valve, and fixing the valve subcutaneously.

11. Method according to claim 9, comprising the alternative step of:
H′) connecting the inflation catheter to the multi-puncture valve by moving it to a side, thus positioning the valve in a site farther from the fixing elements, making another small incision in the skin and tunneling the cellular tissue in order to pass the catheter and connect the same to the valve.
